(19) United States
(12) Patent Application Publication
(10) Pub. No.: US 2019/0374366 A1
(43) Pub. Date: Dec. 12, 2019

(54) BELT WITH INTERNAL DISTRACTION

(71) Applicant: JO Health LLC, Miami Beach, FL (US)

(72) Inventor: Jesse W. Ohliger, Miami Beach, FL (US)

(73) Assignee: JO Health LLC, Miami Beach, FL (US)

(21) Appl. No.: 16/434,769

(22) Filed: Jun. 7, 2019

Related U.S. Application Data
(60) Provisional application No. 62/681,837, filed on Jun. 7, 2018.

(51) Int. Cl. A61F 5/02; A61F 5/28; A61F 5/34

(52) U.S. Cl. CPC A61F 5/028; A61F 5/34; A61F 5/28

(57) ABSTRACT

According to one example, a belt includes an inner belt configured to encircle a waist portion of a user, a first set of one or more pockets coupled to a first position on an internal side of the inner belt, and a second set of one or more pockets coupled to a second position on the internal side. The first set of pockets are configured to receive a first set of one or more distraction devices, wherein the first position is configured to cause the first set of distraction devices to apply pressure to one or more abdominal portions of the user. The second set of pockets are configured to receive a second set of one or more distraction devices, wherein the second position is configured to cause the second set of distraction devices to apply pressure to one or more lower back portions of the user.

### CROSS-REFERENCE TO RELATED APPLICATIONS

This application claims priority to U.S. Provisional Patent Application No. 62/681,837 entitled “Belt With Internal Distraction” and filed Jun. 7, 2018, the entirety of which is incorporated herein by reference.

### TECHNICAL FIELD

This disclosure relates generally to the field of fitness training devices/physical therapy device and more specifically to a belt with internal distraction.

### BACKGROUND

The majority of adults live a sedentary lifestyle. This leads to tight or weak abdominal and lumbothoracic muscles, poor hip mobility, and hip stability, which will ultimately affect an individual's ability to breathe diaphragmatically and regulate physical and emotional stress.

The muscular system of hip and spine function are deeply connected to the mechanics of the respiratory system and function. Both systems must function properly in order to regulate the body's response to daily stressors of everyday life.

An estimated 90% of adults cannot properly diaphragmatically breathe (i.e., referred to as a “belly breath”). This is not surprising, as each day the average adult typically spends 11-12 hours in a seated position and 6-7 hours sleeping laying down.

As an individual's lifestyle becomes sedentary, a person's hips are placed in a constant state of flexion. This causes abdominal and lumbothoracic tissues to become tight or weak, restricting the function of the deeper lumbar stabilizing muscles, the iliopsoas, and Quadratus Lumborum that are connected to the diaphragm fascia. When the diaphragm becomes restricted it may affect one's ability to create a balance of intra-abdominal pressure in both movement and communication.

A common posture issue synonymous with tight hip flexors is known as anterior pelvic tilt. A person that has an anterior pelvic tilt typically has a weak or shortened iliopsoas, which may cause dysfunction in gluteal muscles, hamstrings, and adductors. The iliopsoas muscle may be the most important muscle in a person's body. It is vital to survival as it is responsible for hip flexion, and it plays an integral role in the respiratory and central nervous systems, among others. It is the only muscle that connects the upper body (12th thoracic vertebrae) to the lower body (femur). The iliopsoas also helps stabilize a person's hips and spine, and it is connected to the diaphragm, which is vital for respiratory function. A sedentary lifestyle often leads to a weak or shortened iliopsoas muscle.

### SUMMARY

According to one example, a belt includes an inner belt configured to encircle a waist portion of a user, a first set of one or more pockets coupled to a first position on an internal side of the inner belt, and a second set of one or more pockets coupled to a second position on the internal side. The first set of pockets are configured to receive a first set of one or more distraction devices, wherein the first position is configured to cause the first set of distraction devices to apply pressure to one or more abdominal portions of the user. The second set of pockets are configured to receive a second set of one or more distraction devices, wherein the second position is configured to cause the second set of distraction devices to apply pressure to one or more lower back portions of the user.

According to another example, a method includes positioning an inner belt around a waist portion of a user. The inner belt includes a first end, a second end, and a length that extends from the first end to the second end. The method further includes coupling the first end of the inner belt to the second end of the inner belt so that the inner belt encircles the waist portion of the user, positioning a first set of one or more distraction devices into a first set of one or more pockets coupled to the inner belt, so as to cause the first set of one or more distraction devices to apply pressure to one or more abdominal portions of the user when the inner belt encircles the waist portion of the user, and positioning a second set of one or more distraction devices into a second set of one or more pockets coupled to the inner belt, so as to cause the second set of one or more distraction devices to apply pressure to one or more lower back portions of the user when the inner belt encircles the waist portion of the user.

According to a further example, a method includes coupling a first set of one or more pockets to a first position on an internal side of an inner belt configured to encircle a waist portion of a user. The first set of one or more pockets are configured to receive a first set of one or more distraction devices. The first position of the first set of one or more pockets on the internal side of the inner belt is configured to cause the first set of one or more distraction devices to apply pressure to one or more abdominal portions of the user when the inner belt encircles the waist portion of the user. The internal side of the inner belt is configured to face the user when the inner belt encircles the waist portion of the user. The method further includes coupling a second set of one or more pockets to a second position on the internal side of the inner belt. The second set of one or more pockets are configured to receive a second set of one or more distraction devices. The second position of the second set of one or more pockets on the internal side of the inner belt is configured to cause the second set of one or more distraction devices to apply pressure to one or more lower back portions of the user when the inner belt encircles the waist portion of the user.

### BRIEF DESCRIPTION OF THE FIGURES

For a more complete understanding of the present disclosure and its features and advantages, reference is now made to the following description, taken in conjunction with the accompanying drawings, in which:

FIG. 1 illustrates an example belt system;

FIG. 2 illustrates an example of the positioning of distraction device(s) 46 of the belt system of FIG. 1;

FIG. 3 illustrates another example of the positioning of distraction device(s) 62 of the belt system of FIG. 1;

FIG. 4 illustrates a front view of the belt system of FIG. 1;

FIG. 5 illustrates a back view of the belt system of FIG. 1;

FIG. 6 illustrates a top view of the belt system of FIG. 1; and

FIG. 7 illustrates a front view of the belt system of FIG. 1, with the first end and the second end of the inner belt coupled together and the first end and the second end of the outer belt coupled together.

### DETAILED DESCRIPTION

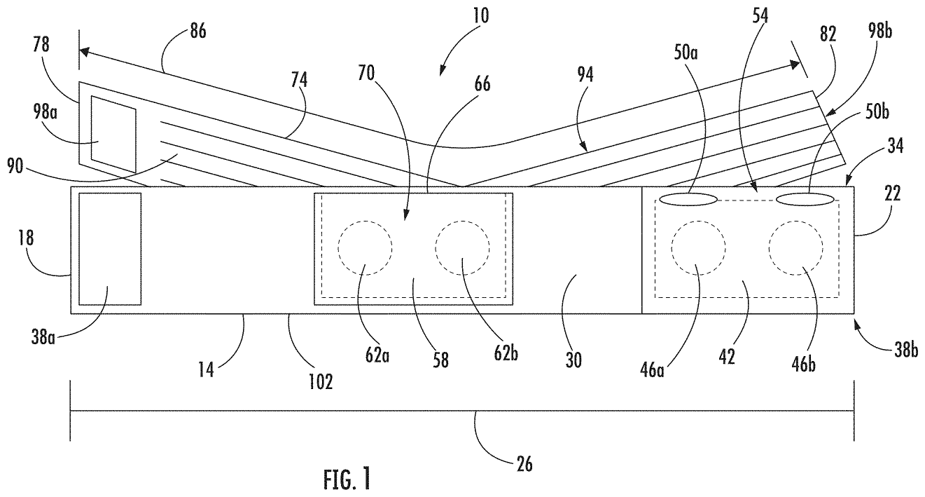

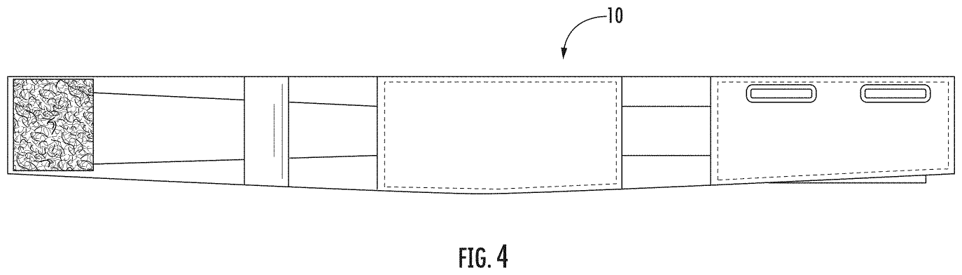

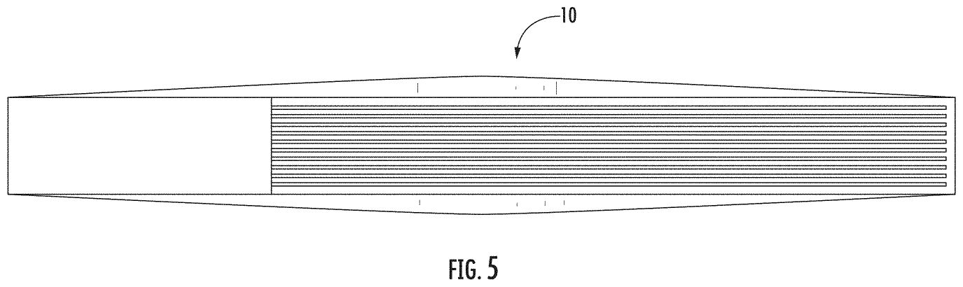

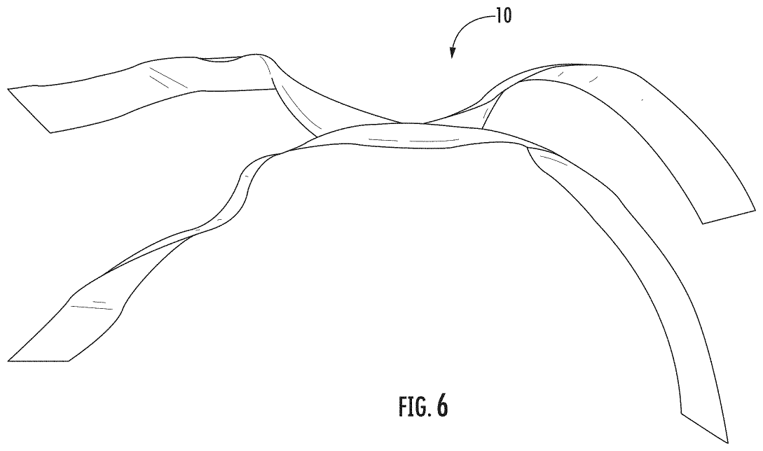

Embodiments of the present disclosure are best understood by referring to FIGS. 1-7 of the drawings, like numerals being used for like and corresponding parts of the various drawings.

FIGS. 1 and 4-7 illustrate an example belt system 10 that a user may encircle around their waist, so as to wear the belt system 10. As is discussed below, the example belt system 10 includes pockets 42 and 58 into which one or more distraction devices 46 and 62 may be positioned. When positioned in the pockets 42 and 58, these distraction devices 46 and 62 may apply internal distraction to the user when the belt system 10 is being worn by the user. This internal distraction may be in the form of pressure that is applied to portions of the user's body. For example, this internal distraction may be in the form of pressure that is applied to the user's left and right abdominal oblique muscles, and it also may be in the form of pressure that is applied to the user's left and right sacroiliac joints. This may, in some examples, help train the user to utilize proper diaphragmatic breathing patterns (i.e., belly breaths). Alternatively, or additionally, the belt system 10 may strengthen the muscles that support breath capacity, control, and coordination, with each inhale and exhale of the user, in some examples. Alternatively, or additionally, the belt system 10 may help unrestrict the user's iliopsoas. For example, the internal distraction may actively lengthen the user's hip flexors, obliques, and transverse abdominal muscle (TrA) fascia, relieving some of the restriction of the user's iliopsoas and/or decompressing the vertebrae of the user's lumbar spine, in some examples. Alternatively, or additionally, the belt system 10 may improve hip mobility and stability. For example, the internal distraction may relieve the superior and inferior gluteal nerves, freeing restriction signal from the central nervous system (CNS) to turn on the gluteus maximus, gluteus medius and the gluteus minimus—thereby providing improvement in stability and mobility.

As is illustrated, the belt system 10 includes an inner belt 14 configured to encircle the waist portion of a user (i.e., where a user refers to any person, including males, females, adults, and/or children). The inner belt 14 includes a first end 18, a second end 22, and a length 26 that extends from the first end 18 to the second end 22. The length 26 may be any length that allows the inner belt 14 to encircle the waist portion of the user. The inner belt 14 further includes an internal side 30 and an external side 34, with the external side 34 being opposite of the internal side 30. The internal side 30 is configured to face the user when the inner belt 14 encircles the waist portion of the user. The external side 34 is configured to face away from the user when the inner belt 14 encircles the waist portion of the user.

The inner belt 14 may be made of any material that allows the inner belt 14 to be encircled around the waist portion of the user. As an example, the inner belt 14 may be made of a stretchable fabric (e.g., made of nylon and/or polyester) that allows the inner belt 14 to stretch and move when the inner belt 14 is encircled around the waist portion of the user. The inner belt 14 may further include one or more ribs positioned within the material of the inner belt 14. For example, the inner belt 14 may include plastic or metal ribs (or stays) that may provide additional support to a user's back when the inner belt 14 encircles the waist portion of the user. These ribs may be positioned within the material of the inner belt 14 at a location similar to the location of the back pocket(s) 58 discussed below.

The inner belt 14 further includes one or more connectors 38 (e.g., 38a and 38b) that may couple the first end 18 to the second end 22 in order to keep the inner belt 14 encircled around a waist portion of the user. A connector 38 may be any device that may couple the first end 18 to the second end 22. For example, as is illustrated, connector 38a may be a piece of velcro coupled on the internal side 30 of the inner belt 14 at a location adjacent the first end 18, while connector 38b may be another corresponding piece of velcro coupled on the external side 34 of the inner belt 14 at a location adjacent the second end 22. These velcro pieces may be attached to each other, thereby coupling the first end 18 to the second end 22. As other examples, the connector 38 may be a zipper, a hook, a belt buckle system, any other device that may couple the first end 18 to the second end 22, or any combination of the preceding. When coupled together, the first end 18 of the inner belt 14 may overlap the second end 18 of the inner belt 14.

The belt system 10 further includes one or more front pockets 42 coupled to the internal side 30 of the inner belt 14. A front pocket 42 may be referred to as “front” pocket 42 because it may be positioned on the inner belt 14 in a location that faces the front of a user when the inner belt 14 encircles the waist portion of the user. The front pocket 42 may be any type of pocket or device configured to receive and hold one or more distraction devices 46. Furthermore, the front packet 42 may further be any type of pocket or device configured to allow the distraction devices 46 to be removed from the front pocket 42.

The belt system 10 may include any number of front pockets 42. For example, the belt system 10 may include one front pocket 42, two front pockets 42, three front pockets 42, four front pockets 42, or any other number of front pockets 42. Also, a front pocket 42 may have any shape and/or size that allows it to receive and hold one or more distraction devices 46. As is illustrated, the belt system 10 includes a single front pocket 42 having a rectangular shape that is sized to receive and/or hold at least two distraction devices 46. The single front pocket 42 also includes two openings 50 (e.g., 50a and 50b) that allow the distraction devices 46 to be inserted and removed from the front pocket 42. Furthermore, although front pocket 42 is illustrated as a single rectangular shaped pocket that holds at least two distraction devices 46, in other examples, the front pocket 42 may be broken up into two individual front pockets 42 that each hold a single distraction device 46.

The front pocket 42 is coupled to the internal side 30 of the inner belt 14. The front pocket 42 may be coupled to the internal side 30 of the inner belt 14 in any manner. For example, as is illustrated, the front packet 42 may be an additional piece of fabric (or other material) that is sewn to the internal side 30 of the inner belt 14. As other examples, the front pocket 42 may be clipped to the internal side 30 of the inner belt 14, zipped to the internal side 30 of the inner belt 14, formed integral with the internal side 30 of the inner belt 14, coupled in any other manner to the internal side 30 of the inner belt 14, or any combination of the preceding. When formed integral with the internal side 30 of the inner belt 14, the front pocket 42 may be positioned in the material of the inner belt 14. By being coupled to the internal side 30 of the inner belt 14 (as opposed to the external side 34), the front pocket(s) 42 and any distraction device(s) 46 positioned within the pocket(s) 42 may be able to apply additional internal distraction to the user when the inner belt 14 encircles the waist portion of the user, in some examples.

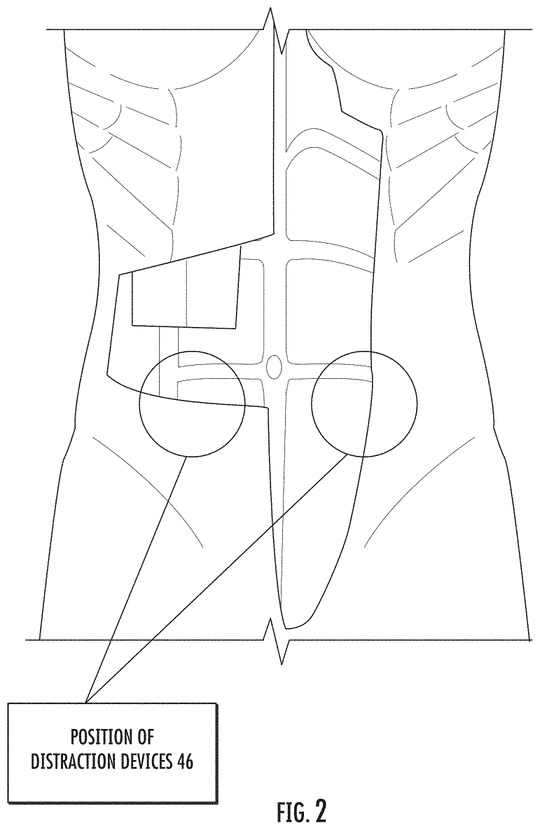

The front pocket 42 may be coupled to the internal side 30 of the inner belt 14 at a first position 54. This first position 54 may be selected so as to cause distraction devices 46 (positioned in the front pocket 42) to apply pressure to one or more abdominal portions of the user when the inner belt 14 encircles the waist portion of the user. For example, this positioning may cause a distraction device 46 (e.g., distraction device 46a) to apply pressure to the left abdominal oblique muscle of the user, and cause another distraction device 46 (e.g., distraction device 46b) to apply pressure to the right abdominal oblique muscle of the user. This applied pressure may track the iliopsoas of the user, in some examples. An example of where this pressure is applied is seen in FIG. 2, which illustrates the position of distraction devices 46 in relation to the user. The first position 54 may be located at any position along the length 26 that allows the distraction device(s) 46 positioned within the front pocket(s) 42 to apply pressure to one or more abdominal portions of the user. As is illustrated, the first position 54 is located adjacent the second end 22 of the inner belt 14.

The belt system 10 further includes one or more distraction devices 46. A distraction device 46 may include any device or item that may apply pressure to a portion of a user when the distraction device 46 is positioned within a front pocket 42 and also when the inner belt 14 encircles the waist portion of the user. As an example, a distraction device 46 may be any device or item having a size and/or shape that causes it to extend inward towards a user from the internal side 30 of the inner belt 14. The distraction device 46 may have any size and/or shape that causes it to extend inward towards the user.

Examples of the distraction device 46 include padding, clothing (e.g., a sock, a folded sock), air pressure tubing, a ball (e.g., a tennis ball, a racquet ball, a myofascial release ball, a pressurized ball), a pad, any other device or item that may apply pressure to a portion of a user when the distraction device 46 is positioned within a front pocket 42 and also when the inner belt 14 encircles the waist portion of the user, or any combination of the preceding. In some examples, the distraction device 46 may be a static device (e.g., it may not move or vibrate). In other examples, the distraction device 46 may be a dynamic device (e.g., it may move or vibrate). For example, the distraction device 46 may be a vibrating pad or ball. In some examples, the distraction device 46 may be heated above room temperature or cooled below room temperature. As is illustrated, the distraction devices 46a and 46b are myofascial release balls.

The belt system 10 further includes one or more back pockets 58 coupled to the internal side 30 of the inner belt 14. A back pocket 58 may be referred to as “back” pocket 58 because it may be positioned on the inner belt 14 in a location that faces the back of a user when the inner belt 14 encircles the waist portion of the user. The back pocket 58 may be any type of pocket or device configured to receive and hold one or more distraction devices 62. Furthermore, the back pocket 58 may further be any type of pocket or device configured to allow the distraction devices 62 to be removed from the back pocket 58.

The belt system 10 may include any number of back pockets 58. For example, the belt system 10 may include one back pocket 58, two back pockets 58, three back pockets 58, four back pockets 58, or any other number of back pockets 58. Also, a back pocket 58 may have any shape and/or size that allows it to receive and hold one or more distraction devices 62. As is illustrated, the belt system 10 includes a single back pocket 58 having a rectangular shape that is sized to receive and/or hold at least two distraction devices 62. The single back pocket 58 also includes a single opening 66 (e.g., in the top) that allows the distraction devices 62 to be inserted and removed from the back pocket 58. Furthermore, although back pocket 58 is illustrated as a single rectangular shaped pocket that holds at least two distraction devices 62, in other examples, the back pocket 58 may be broken up into two (or more) individual back pockets 58 that each hold a single distraction devices 62. Additionally, although back pocket 58 is illustrated as only including a single opening 66, it may include a separate opening 66 for each distraction device 62 (e.g., similar to openings 50 of front pocket 42).

The back pocket 58 is coupled to the internal side 30 of the inner belt 14. The back pocket 58 may be coupled to the internal side 30 of the inner belt 14 in any manner. For example, as is illustrated, the back pocket 58 may be an additional piece of fabric (or other material) that is sewn to the internal side 30 of the inner belt 14. As other examples, the back pocket 58 may be clipped to the internal side 30 of the inner belt 14, zipped to the internal side 30 of the inner belt 14, formed integral with the internal side 30 of the inner belt 14, coupled in any other manner to the internal side 30 of the inner belt 14, or any combination of the preceding. When formed integral with the internal side 30 of the inner belt 14, the back pocket 58 may be positioned in the material of the inner belt 14. By being coupled to the internal side 30 of the inner belt 14 (as opposed to the external side 34), the back pocket(s) 58 and any distraction device(s) 62 positioned within the pocket(s) 58 may be able to apply additional internal distraction to the user when the inner belt 14 encircles the waist portion of the user, in some examples.

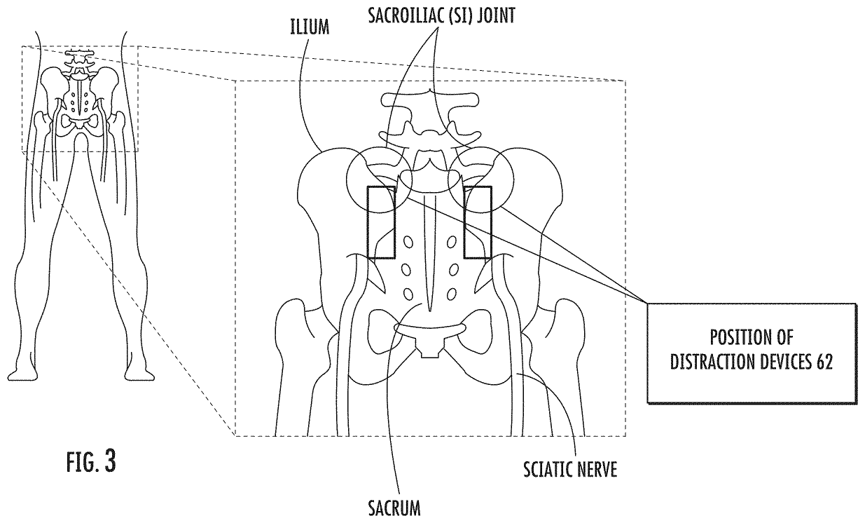

The back pocket 58 may be coupled to the internal side 30 of the inner belt 14 at a second position 70. This second position 70 may be selected so as to cause distraction devices 62 (positioned in the back pocket 58) to apply pressure to one or more lower back portions of the user when the inner belt 14 encircles the waist portion of the user. For example, this positioning may cause a distraction device 62 (e.g., distraction device 62a) to apply pressure to the right sacroiliac joint of the user, and cause another distraction device 62 (e.g., distraction device 62b) to apply pressure to the left sacroiliac joint of the user of the user. An example of where this pressure is applied is seen in FIG. 3, which illustrates the position of distraction devices 62 in relation to the user. The second position 70 may be located at any position along the length 26 that allows the distraction device(s) 62 positioned within the back pocket(s) 58 to apply pressure to one or more lower back portions of the user. As is illustrated, the second position 70 is located at a half-way point along the length 26.

The belt system 10 further includes one or more distraction devices 62. A distraction device 62 may include any device or item that may apply pressure to a portion of a user when the distraction device 62 is positioned within a back pocket 58 and also when the inner belt 14 encircles the waist portion of the user. As an example, a distraction device 62 may be any device or item having a size and/or shape that causes it to extend inward towards a user from the internal side 30 of the inner belt 14. The distraction device 62 may have any size and/or shape that causes it to extend inward towards the user.

Examples of the distraction device 62 include padding, clothing (e.g., a sock, a folded sock), air pressure tubing, a ball (e.g., a tennis ball, a racquet ball, a myofascial release ball, a pressurized ball), a pad, any other device or item that may apply pressure to a portion of a user when the distraction device 62 is positioned within a back pocket 58 and also when the inner belt 14 encircles the waist portion of the user, or any combination of the preceding. In some examples, the distraction device 62 may be a static device (e.g., it may not move or vibrate). In other examples, the distraction device 62 may be a dynamic device (e.g., it may move or vibrate). For example, the distraction device 62 may be a vibrating pad or ball. In some examples, the distraction device 62 may be heated above room temperature or cooled below room temperature. As is illustrated, the distraction devices 62a and 62b are myofascial release balls.

The distraction device 62 may be the same type of device or item as the distraction device 46. For example, as is illustrated, the distraction devices 46 and 62 may all be myofascial release balls. Alternatively, the distraction device 62 may be a different type of device or item than the distraction device 46. For example, the distraction devices 46 may be myofascial release balls, while the distraction devices 62 may be folded socks. Any combination of types of distraction device(s) 46 and distraction device(s) 62 may be used in the belt system 10.

As is illustrated, the belt system 10 includes an outer belt 74 configured to encircle the waist portion of the user and further configured to encircle the inner belt 14 when the inner belt 14 encircles the waist portion of the user. The outer belt 74 includes a first end 78, a second end 82, and a length 86 that extends from the first end 78 to the second end 82. The length 86 may be any length that allows the outer belt 74 to encircle the waist portion of the user and encircle the inner belt 14. The outer belt 74 further includes an internal side 90 and an external side 94, with the external side 94 being opposite of the internal side 90. The internal side 90 is configured to face the user when the outer belt 74 encircles the waist portion of the user. The external side 94 is configured to face away from the user when the outer belt 74 encircles the waist portion of the user.

The outer belt 74 may be made of any material that allows the outer belt 74 to be encircled around the waist portion of the user. As an example, the outer belt 74 may be made of an elastic material (e.g., made of nylon and/or polyester) that allows the outer belt 74 to stretch and move when the outer belt 74 is encircled around the waist portion of the user. The outer belt 74 may be made of multiple layers. For example, the outer belt 74 may be made of multiple layers that are attached together to increase the strength of the outer belt 74.

As is illustrated, the outer belt 74 is coupled to the external side 34 of the inner belt 14. The outer belt 74 may be coupled to the external side 34 of inner belt 14 in any manner. For example, as is illustrated, the outer belt 74 may be sewn to the external side 34 of the inner belt 14. As other examples, the outer belt 74 may be clipped to the external side 34 of the inner belt 14, zipped to the external side 34 of the inner belt 14, coupled in any other manner to the external side 34 of the inner belt 14, or any combination of the preceding. In some examples, as is illustrated in FIG. 1, the outer belt 74 is coupled to the inner belt 14 in a V-formation, where the half-way point of the outer belt 74 is coupled to the inner belt 14. This may allow the first end 74 and second end 82 of the outer belt 74 to move freely from the inner belt 14, while still remaining coupled to the inner belt 14.

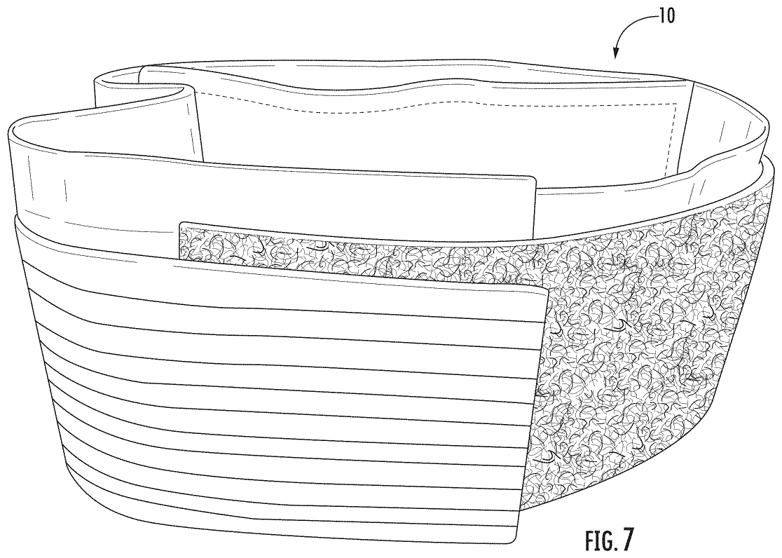

The outer belt 74 further includes connectors 98 (e.g., 98a and 98b) that may couple the first end 78 to the second end 82 in order to keep the outer belt 74 encircled around a waist portion of the user. A connector 98 may be any device that may couple the first end 78 to the second end 82. For example, as is illustrated, connector 98a may be a piece of velcro coupled on the internal side 90 of the outer belt 74 at a location adjacent the first end 78, while connector 98b may be another corresponding piece of velcro coupled on the external side 94 of the outer belt 74 at a location adjacent the second end 82. These velcro pieces may be attached to each other, thereby coupling the first end 78 to the second end 82. As other examples, the connector 98 may be a zipper, a hook, a belt buckle system, any other device that may couple the first end 78 to the second end 82, or any combination of the preceding. When coupled together, the first end 78 of the outer belt 74 may overlap the second end 82 of the outer belt 74. FIG. 7 illustrates an example of the belt system 10 when the first end 18 and the second end 22 of the inner belt 14 are coupled to each other, and when the first end 78 and the second end 82 of the outer belt 74 are coupled to each other.

Modifications, additions, and/or substitutions may be made to the belt system 10, the components of the belt system 10, and/or the functions of the belt system 10 without departing from the scope of the specification. For example, although the belt system 10 is illustrated in FIG. 1 as including outer belt 74, in some examples, the belt system 10 may not include the outer belt 74. In examples where the belt system 10 includes the outer belt 74, the outer belt 74 may increase the amount of internal distraction applied to a user of the belt system 10. For example, the outer belt 74 may apply pressure to the inner belt 14 and the distraction devices 46 and 62 positioned in the pockets 42 and 58 of the inner belt 14, causing an increase in internal distraction applied to the user. As a further example, although the outer belt 74 is illustrated in FIG. 1 as being coupled to the inner belt 14, in some examples, they may be two separate belts that are only coupled together when they both encircle a user's waist portion.

As another example, although the belt system 10 is illustrated in FIG. 1 as including front pocket(s) 42 and back pocket(s) 58, the belt system 10 may only include front pocket(s) 42 (and not back pocket(s) 58). Alternatively, the belt system 10 may only include back pocket(s) 58 (and not front pocket(s) 42). Furthermore, the pocket(s) 42 and/or pocket(s) 58 may be moveable on the belt system 10. For example, the inner belt 14 may include a rail system along the length 26, allowing the pocket(s) 42 and/or pocket(s) 58 to be slid along the length 26 to any position. This may allow internal distraction to be applied to different areas of the user. As another example, the inner belt 14 may include velcro patches (or other connectors) along its length 26, allowing pocket(s) 42 and/or pocket(s) 58 to be removed from a first location and moved to a second location (or removed entirely from the belt system 10). Additionally, in some examples, the pocket(s) 42 and/or pocket(s) 58 may be positioned in different (or additional) locations on the belt system 10. As an example, the pocket(s) 42 and/or pocket(s) 58 may be positioned so that distraction device(s) 46 and/or 62 apply pressure between a person's bottom rib and the person's hip on either side of the person's body (obliques) where the rib cage is closest to the hip bones (iliums).

As a further example, although the distraction device(s) 46 and 62 are described above as being removable from the pocket(s) 42 and pocket(s) 58, in some examples, the distraction device(s) 46 and 62 may be permanently positioned within the pocket(s) 42 and pocket(s) 58. For example, the distraction device(s) 46 and 62 may be sewn into the pocket(s) 42 and pocket(s) 58. As another example, the distraction device(s) 46 and 62 may be sewn into the inner belt 14 of the belt system 10 (e.g., as internal mounds). Such a permanent position may prevent the distraction device(s) 46 and 62 from being removed from the belt system 10 without damaging the belt system 10.

In an exemplary embodiment of operation of belt system 10, the user may desire to wear the belt system 10 so as to receive the internal distraction provided by the belt system 10. In order to do so, the user may pick up the inner belt 14 and may position the inner belt 14 around the user's waist portion. In such an example, the user may position the inner belt 14 with the internal side 30 of the inner belt 14 facing the user. Additionally, the user may position the inner belt 14 so that the bottom edge 102 of the inner belt 14 is positioned approximately 2 inches below the top of the user's hips. This may cause the inner belt 14 to cover the top 2 inches of the user's hips.

Once positioned around the user's waist portion, the user may couple the first end 18 of the inner belt 14 to the second end 22 of the inner belt 14 so that the inner belt 14 encircles the waist portion of the user. To do so, the user may couple connector 38a on the internal side 30 of the inner belt 14 to the connector 38b on the external side 34 of the inner belt 14. In some examples, this may be accomplished by pulling the second end 22 of the inner belt 14 across the waist of the user, and then pulling the first end 18 of the inner belt 14 across the waist of the user in the opposite direction. The connector 38a (e.g., velcro) may then be attached to the connector 38b (e.g., velcro) to encircle the waist portion of the user. In some examples, the user may inhale prior to and while coupling the first end 18 to the second end 22. This may allow the inner belt 14 to be more tightly encircled around the waist portion of the user. In some examples, by pulling the second end 22 of the inner belt 14 across the waist of the user, the front pocket(s) 42 may be positioned against the front of the user's waist. As is discussed below, this may allow the distraction devices 46 positioned within the front pocket 46 to apply pressure to one or more abdominal portions of the user.

The user may be standing when the first end 18 is coupled to the second end 22. In other examples, the user may be laying down on their back when the first end 18 is coupled to the second end 22. When laying down, the user may position their legs up against a wall, with their knees bent at approximately a 90° angle.

Following the coupling of the first end 18 to the second end 22 so that the inner belt 14 encircles the waist portion of the user, the user may position one or more distraction devices 46 into one or more front pockets 42. For example, the user may position two distraction devices 46 (e.g., balls) into a single front pocket 42, with each being positioned through a respective opening 50a and 50b. By positioning the distraction device(s) 46 into the front pocket 42, each distraction device 46 may be positioned halfway between the user's belly button and the user's anterior superior iliac spine. For example, the distraction device 46a may be positioned south and left of the user's belly button in a location that is halfway between the user's belly button and the user's anterior superior iliac spine (e.g., placed from the anterior superior iliac spine diagonally up the spine on the front abdominals up towards the middle of the rib cage), while the distraction device 46b may be positioned south and right of the user's belly button in a location that is halfway between the user's belly button and the user's anterior superior iliac spine (e.g., placed from the anterior superior iliac spine diagonally up the spine on the front abdominals up towards the middle of the rib cage). This positioning may be where the rectus abdominus and obliques of the user meet. An example of this positioning is illustrated in FIG. 2.

The positioning of the distraction devices 46 may cause the distraction devices 46 to apply pressure to one or more abdominal portions of the user. In some examples, the distraction device 46a may apply pressure to the left abdominal oblique muscle, and the distraction device 46b may apply pressure to the right abdominal oblique muscle of the user. By doing so, the distraction devices 46 may apply pressure at a location that tracks the user's iliopsoas.

Also following the coupling of the first end 18 to the second end 22 so that the inner belt 14 encircles the waist portion of the user, the user may position one or more distraction devices 62 into one or more back pockets 58. For example, the user may position two distraction devices 62 (e.g., balls) into a single back pocket 58, with each being positioned through the opening 66. This positioning may cause the distraction devices 62 to apply pressure to one or more lower back portions of the user. In some examples, the distraction device 62a may apply pressure to the right sacroiliac joint of the user (e.g., the top portion of the right sacroiliac joint), and the distraction device 62b may apply pressure to the left sacroiliac joint of the user (e.g., the top portion of the left sacroiliac joint). An example of this positioning of the distraction devices 46 (and the pressure applied by this positioning) is illustrated in FIG. 3. In some examples, the application of pressure to the right sacroiliac joint/left sacroiliac joint may refer to pressure being applied to the lower back portion of the user in a location that is within 1 inch of the top portion of the right sacroiliac joint/left sacroiliac joint.

Following positioning of the distraction devices 46 and 62, the user may position the outer belt 74 around the user's waist portion. In such an example, the user may position the outer belt 74 with the internal side 90 of the inner belt 14 facing the user. Once positioned around the user's waist portion, the user may couple the first end 78 of the outer belt 74 to the second end 82 of the outer belt 74 so that the outer belt 74 encircles the waist portion of the user and further encircles the inner belt 14. To do so, the user may couple connector 98a on the internal side 90 of the outer belt 74 to the connector 98b on the external side 94 of the outer belt 74. In some examples, this may be accomplished by pulling the second end 82 of the outer belt 74 across the waist of the user, and then pulling the first end 78 of the outer belt 74 across the waist of the user in the opposite direction. The connector 98a (e.g., velcro) may then be attached to the connector 98b (e.g., velcro) to encircle the waist portion of the user and encircle the inner belt 14. In some examples, the user may inhale prior to and while coupling the first end 78 to the second end 82. This may allow the outer belt 74 to be more tightly encircled around the waist portion of the user.

The user may be standing when the first end 78 is coupled to the second end 82. In other examples, the user may be laying down on their back when the first end 78 is coupled to the second end 82. When laying down, the user may position their legs up against a wall, with their knees bent at approximately a 90° angle.

Following the above set-up of the belt system 10, the user may begin to utilize the belt system 10. For example, when the user inhales while wearing the belt system 10, the user's belly may expand into the inner belt 14, causing the distraction devices 46 to be pushed (or feel like they are being pushed) into the user's belly (while the distraction devices 62 may be pushed (or feel like they are being pushed) into the user's back). This pressure may be relieved (at least partially) when the user subsequently exhales. In some examples, this pressure may notify the user that they are currently breathing using a proper belly breath technique. For example, the distraction devices 46 and 62 may initiate a neurological feedback loop from the skin, muscles and fascia of the user, bringing a heighted sense of awareness to that area, along with the physiological changes to the user's body. Thus, in some examples, the belt system 10 may train the user to breathe using belly breaths. To further assist in such training, the user may focus on trying to push the distraction devices 46 and 62 out (e.g., horizontally out) when inhaling, and trying to allow the distraction devices 46 and 62 to sink further into the user's body (e.g., horizontally in) when exhaling.

Alternatively, or additionally, use of the belt system 10 may strengthen the muscles that support breath capacity, control, and coordination, with each inhale and exhale of the user, in some examples. Alternatively, or additionally, use of the belt system 10 may help unrestrict the user's iliopsoas. For example, the internal distraction provided by the belt system 10 may actively lengthen the user's hip flexors, obliques, and TrA fascia, relieving some of the restriction of the user's iliopsoas and/or decompressing vertebrae of the user's lumbar spine, in some examples. Alternatively, or additionally, use of the belt system 10 may improve hip mobility and stability. For example, the internal distraction provided by the belt system 10 may relieve to the superior and inferior gluteal nerves, freeing restriction signal from the CNS to turn on the gluteus maximus, gluteus medius and the gluteus minimus—thereby providing improvement in stability and mobility.

One or more benefits of using the belt system 10 may include (but may not be limited to): (1) active firing of iliopsoas, lateral obliques, pelvic floor; (2) improved glute patterning, function and stability, hip mobility; (3) compress and support sacroiliac joints; (4) decompression in lumbar/thoracic spine; (5) improved breath control and capacity; (6) relieves back pain; (7) improves posture; (8) reduced rib flair; and/or (9) catalyst to teach “hip hinge” to athlete or patient for improved hip mobility.

The belt system 10 may be utilized by a user at any time. For example, the belt system 10 may be worn while laying down, while exercising (e.g., walking, running, playing golf, weight training, yoga, pilates, martial arts, crossfit, speed and agility training, etc.), while driving (e.g., in a car), while flying (e.g., in a plane), while performing rehabilitation exercises, after the user is finished with a physical therapy session or a chiropractic session, any other time, or any combination of the preceding. Additionally, the belt system 10 may, in some examples, assist in post-natal rehabilitation, injury prevention, all sports or movement practices, reduction of back pain, scoliosis aid, personal training aid, helping patients at a chiropractic and/or physical therapy office, or any combination of the preceding.

Modifications, additions, and/or substitutions may be made to the example steps discussed above. For example, although the distraction devices 46 and 62 are described above as being positioned after the inner belt 14 is encircled around the user's waist portion, the distraction devices 46 and 62 may be positioned in the pockets 42 and 58 before the inner belt 14 is positioned around the waist of the user, or at any other time. Furthermore, one or more of the distraction devices 46 and/or distraction devices 62 may be removed or replaced at any time. For example, if a first set of distraction devices 46 (e.g., balls) are providing too much internal distraction (e.g., pressure), they may be removed and replaced at any time with a different set of distraction devices 46 (e.g., folded socks, smaller balls).

As another example, the steps discussed above may include additional steps or fewer steps. As an example, the belt system 10 may not include an outer belt 74. In such an example, the steps discussed above regarding the outer belt 74 may not be performed.

This specification has been written with reference to various non-limiting and non-exhaustive embodiments or examples. However, it will be recognized by persons having ordinary skill in the art that various substitutions, modifications, or combinations of any of the disclosed embodiments or examples (or portions thereof) may be made within the scope of this specification. Thus, it is contemplated and understood that this specification supports additional embodiments or examples not expressly set forth in this specification. Such embodiments or examples may be obtained, for example, by combining, modifying, or reorganizing any of the disclosed steps, components, elements, features, aspects, characteristics, limitations, and the like, of the various non-limiting and non-exhaustive embodiments or examples described in this specification. In this manner, Applicant reserves the right to amend the claims during prosecution to add features as variously described in this specification.

What is claimed is:

1. A belt, comprising:
an inner belt configured to encircle a waist portion of a user, the inner belt comprising:
a first end, a second end, and a length that extends from the first end to the second end;
an internal side configured to face the user when the inner belt encircles the waist portion of the user; and
an external side configured to face away from the user when the inner belt encircles the waist portion of the user;
a first set of one or more pockets coupled to a first position on the internal side of the inner belt, the first set of one or more pockets configured to receive a first set of one or more distraction devices, wherein the first position of the first set of one or more pockets on the internal side of the inner belt is configured to cause the first set of one or more distraction devices to apply pressure to one or more abdominal portions of the user when the inner belt encircles the waist portion of the user, wherein the first set of one or more pockets are further configured to allow the first set of one or more distraction devices to be removed from the first set of one or more pockets; and
a second set of one or more pockets coupled to a second position on the internal side of the inner belt, the second set of one or more pockets configured to receive a second set of one or more distraction devices, wherein the second position of the second set of one or more pockets on the internal side of the inner belt is configured to cause the second set of one or more distraction devices to apply pressure to one or more lower back portions of the user when the inner belt encircles the waist portion of the user, wherein the second set of one or more pockets are further configured to allow the second set of one or more distraction devices to be removed from the second set of one or more pockets.

2. The belt of claim 1, further comprising an outer belt coupled to the external side of the inner belt and configured to encircle the waist portion of the user and encircle the inner belt when the inner belt encircles the waist portion of the user.

3. The belt of claim 1, wherein the first position of the first set of one or more pockets on the internal side of the inner belt is configured to cause the first set of one or more distraction devices to apply pressure to a left abdominal oblique muscle and a right abdominal oblique muscle of the user when the inner belt encircles the waist portion of the user.

4. The belt of claim 1, wherein the second position of the second set of one or more pockets on the internal side of the inner belt is configured to cause the second set of one or more distraction devices to apply pressure to a left sacroiliac joint and a right sacroiliac joint of the user when the inner belt encircles the waist portion of the user.

5. The belt of claim 1, wherein the first set of one or more pockets comprises one pocket with two openings into the pocket.

6. The belt of claim 1, wherein the second set of one or more pockets comprises one pocket with a single opening into the pocket.

7. The belt of claim 1, wherein the first set of one or more distraction devices comprises one or more pieces of padding, one or more items of clothing, one or more pieces of air pressure tubing, one or more balls, or a combination thereof.

8. The belt of claim 1, wherein the second set of one or more distraction devices comprises one or more pieces of padding, one or more items of clothing, one or more pieces of air pressure tubing, one or more balls, or a combination thereof.

9. A method, comprising:
positioning an inner belt around a waist portion of a user, the inner belt comprising a first end, a second end, and a length that extends from the first end to the second end;
coupling the first end of the inner belt to the second end of the inner belt so that the inner belt encircles the waist portion of the user;
positioning a first set of one or more distraction devices into a first set of one or more pockets coupled to the inner belt, so as to cause the first set of one or more distraction devices to apply pressure to one or more abdominal portions of the user when the inner belt encircles the waist portion of the user; and
positioning a second set of one or more distraction devices into a second set of one or more pockets coupled to the inner belt, so as to cause the second set of one or more distraction devices to apply pressure to one or more lower back portions of the user when the inner belt encircles the waist portion of the user.

10. The method of claim 9, further comprising:
positioning an outer belt around the waist portion of a user, the outer belt being coupled to the internal belt and comprising a first end, a second end, and a length that extends from the first end to the second end;
coupling the first end of the outer belt to the second end of the outer belt so that the outer belt encircles the waist portion of the user and further encircles the inner belt.

11. The method of claim 9, further comprising positioning the first set of one or more distraction devices into the first set of one or more pockets coupled to the inner belt, so as to cause the first set of one or more distraction devices to apply pressure to a left abdominal oblique muscle and a right abdominal oblique muscle of the user when the inner belt encircles the waist portion of the user.

12. The method of claim 9, further comprising positioning the second set of one or more distraction devices into the second set of one or more pockets coupled to the inner belt, so as to cause the second set of one or more distraction devices to apply pressure to a left sacroiliac joint and a right sacroiliac joint of the user when the inner belt encircles the waist portion of the user.

13. The method of claim 9, wherein the first set of one or more distraction devices comprises one or more pieces of padding, one or more items of clothing, one or more pieces of air pressure tubing, one or more balls, or a combination thereof.

14. The method of claim 9, wherein the second set of one or more distraction devices comprises one or more pieces of padding, one or more items of clothing, one or more pieces of air pressure tubing, one or more balls, or a combination thereof.

15. A method, comprising:
coupling a first set of one or more pockets to a first position on an internal side of an inner belt configured to encircle a waist portion of a user, the first set of one or more pockets configured to receive a first set of one or more distraction devices, wherein the first position of the first set of one or more pockets on the internal side of the inner belt is configured to cause the first set of one or more distraction devices to apply pressure to one or more abdominal portions of the user when the inner belt encircles the waist portion of the user, wherein the internal side of the inner belt is configured to face the user when the inner belt encircles the waist portion of the user; and
coupling a second set of one or more pockets to a second position on the internal side of the inner belt, the second set of one or more pockets configured to receive a second set of one or more distraction devices, wherein the second position of the second set of one or more pockets on the internal side of the inner belt is configured to cause the second set of one or more distraction devices to apply pressure to one or more lower back portions of the user when the inner belt encircles the waist portion of the user.

16. The method of claim 15, further comprising coupling an outer belt to the external side of the inner belt, wherein the outer belt is configured to encircle the waist portion of the user and encircle the inner belt when the inner belt encircles the waist portion of the user, wherein the external side of the inner belt is configured to face away from the user when the inner belt encircles the waist portion of the user.

17. The method of claim 15, wherein the first position of the first set of one or more pockets on the internal side of the inner belt is configured to cause the first set of one or more distraction devices to apply pressure to a left abdominal oblique muscle and a right abdominal oblique to muscle of the user when the inner belt encircles the waist portion of the user.

18. The method of claim 15, wherein the second position of the second set of one or more pockets on the internal side of the inner belt is configured to cause the second set of one or more distraction devices to apply pressure to a left sacroiliac joint and a right sacroiliac joint of the user when the inner belt encircles the waist portion of the user.

19. The method of claim 15, wherein the first set of one or more pockets comprises one pocket with two openings into the pocket.

20. The method of claim 15, wherein the second set of one or more pockets comprises one pocket with a single opening into the pocket.
